(19) United States
(12) Patent Application Publication
(10) Pub. No.: US 2019/0013103 A1
(43) Pub. Date: Jan. 10, 2019

(54) SYSTEM FOR DETERMINING OPTIMAL TREATMENT MODALITY FOR LUNG VOLUME REDUCTION AND RELATED METHODS

(71) Applicant: Uptake Medical Technology Inc., Seattle, WA (US)

(72) Inventors: Erik Henne, Seattle, WA (US); Kyle Koelsch, Seattle, WA (US); Jan Rey Pioquinto, Seattle, WA (US); Robert Barry, Seattle, WA (US)

(21) Appl. No.: 16/129,373

(22) Filed: Sep. 12, 2018

Related U.S. Application Data
(63) Continuation-in-part of application No. 15/927,349, filed on Mar. 21, 2018.
(60) Provisional application No. 62/475,074, filed on Mar. 22, 2017.

(51) Int. Cl. G16H 50/20; A61B 5/00; A61B 34/10

(52) U.S. Cl. CPC G16H 50/20; A61B 2017/00809; A61B 34/10; A61B 5/4848

(57) ABSTRACT

A method and system for assisting a physician determine an optimal treatment modality for performing lung volume reduction on a patient. Optimal treatment modalities are determined based on physician preferences relating to the disease attributes and diagnostic patient information. The treatment modalities and patient data are graphically displayed.

### CROSS-REFERENCE TO RELATED APPLICATIONS

This application is a continuation in part application of patent application Ser. No. 15/927,349, filed Mar. 21, 2018, entitled “SYSTEM FOR DETERMINING OPTIMAL TREATMENT MODALITY FOR LUNG VOLUME REDUCTION AND RELATED METHODS”, which claims benefit of provisional patent application No. 62/475,074, filed Mar. 22, 2017, entitled “SYSTEM FOR DETERMINING OPTIMAL TREATMENT MODALITY FOR LUNG VOLUME REDUCTION AND RELATED METHODS”.

### BACKGROUND OF THE INVENTION

The present invention relates to medical methods and systems for treating chronic obstructive pulmonary disease (COPD), and more specifically to methods and systems for assisting a physician determine optimal treatment modalities to perform lung volume reduction on a patient with COPD.

COPD is a chronic disease of the lungs, in which the destruction of the inner lung structure makes breathing increasingly difficult. While symptoms include shortness of breath, excessive production of sputum, and coughing, many people do not experience any symptoms until the later stages of the disease. There are currently many treatment options for COPD but there is no cure, nor a universal standard.

One treatment option for COPD not manageable by systemic delivery of medication is lung volume reduction. Lung Volume reduction is the removal or collapse of damaged lung tissue, thereby allowing the remaining healthy tissue to expand.

Various treatment modalities are available to perform lung volume reduction including vapor ablation, installation of valves or coils, and application of sealant.

However, it is difficult to determine which treatment modality is best because one treatment modality may be more compatible with a particular patient than another treatment modality based on a various patient factors or characteristics. Additionally, a physician may prefer one type of treatment modality over another in view of her experience, skill, and training relating to the various treatment modalities as well as local availability and reimbursement.

Accordingly, a system and method that overcomes the above mentioned challenges is desirable.

### SUMMARY OF THE INVENTION

The present invention is a method and system for assisting a physician to determine an optimal treatment modality for performing lung volume reduction on a patient.

In embodiments, a computer-implemented method provides a plurality of primary categories of disease attributes amongst COPD patients. A physician preference input is received for at least one of the disease attributes. An output corresponding to an optimal treatment modality is determined. The output is based on the physician preference inputs and diagnostic information relating to a patient or group of patients.

In embodiments, the primary categories of disease attributes include disease heterogeneity, segmental or lobar treatment, fissure integrity, maximum destruction criteria, residual volume, and desired treatment locations.

In embodiments, the method additionally provides a choice of sub or minor categories for each primary category. Heterogeneity, for example, has two sub categories, including Lobar Heterogeneity and Segmental Heterogeneity. Regarding Lobar Heterogeneity, the physician enters the threshold ratio in the disease severity that represents a significant difference between two lobes of the lung. Additionally, in embodiments, the physician can select what represents a significant disease difference in two segments of lung.

In embodiments, a primary category or disease attribute is BLVR approach. If segments within a lobe is heterogeneously diseased, then a segmental treatment approach is often preferred to preserve relatively healthy tissue. If a lobe contains equally diseased segments, or homogeneous lobe, then a lobar treatment approach is often preferred. The physician selects whether lobar or segmental approach is preferred. In embodiments, the physician also selects whether a unilateral or bilateral approach is preferred.

In embodiments, a primary category or disease attribute is fissure integrity or completeness criteria. Fissure integrity score is a strong predictor of the presence of collateral ventilation. Blocking technologies such as valves are known to be ineffective in patients with collateral ventilation. The physician chooses a fissure integrity criteria that quantifies or sets a threshold for collateral ventilation. The system allows the physician to qualify or disqualify certain Lung Volume Reduction technologies such as valve-based programs if the fissure integrity score is too low.

In embodiments, the physician selects the maximum percentage of destroyed lobes considered treatable. Mechanical BLVR technologies such as coils cannot effectively hold down tissue to reduce lung volume if the tissue in the diseased region is largely destroyed.

In one embodiment, if the degree of destruction in the patient(s) lobes from the patient data is higher than the physician's input, certain technologies such as coils are automatically excluded, or lowered in score, and ranking.

In embodiments, data is received corresponding to a specific patient, or group of specific patients, and output is determined in the form of an optimal treatment modality for that patient or patient group. The output is determined based on the physician's preferences and the diagnostic information for the patient(s).

In embodiments, a database of patient data based on the demographics of the physician's typical COPD patients is received, and an output is determined and displayed in the form of one or more charts. In embodiments, the percentage of patients within the recommended treatment modalities is displayed. As described herein, determination of the recommended modalities and percentage of patients within each modality is based on the physician's preference criteria as well as the patient(s) data.

In embodiments, a system for assisting a physician perform a lung volume reduction procedure on a patient comprises a processor operable to: (i) compute disease characteristics for bronchial segments, lung lobes, and lung fissures of the patient based on lung image information of the patient; and (ii) transform the lung image information into a non-anatomical shaped graphic indicative of the relative volume and disease characteristics of the bronchial segments, lung lobes, and fissures of the patient; and a display in communication with the processor and operable to present the graphic.

In embodiments, a method for assisting a physician perform a lung volume reduction procedure on a patient comprises computing disease characteristics for bronchial segments, lung lobes, and lung fissures of the patient based on received lung image information of the patient; computing a non-anatomical shaped graphic indicative of the relative volume of the bronchial segments and lung lobes, and disease characteristics corresponding to the bronchial segments and the lung lobes of the patient; and displaying the graphic.

In embodiments, the step of computing the graphic is performed by transforming the image information of the patient's bronchial segments and lung lobes into the non-anatomical shaped graphic.

In embodiments, the transforming comprises converting the lung image data for each lung into a donut-shaped graphic for each lung.

In embodiments, each donut-shaped graphic comprises an inner ring and an outer ring, and wherein each inner and outer ring comprises a plurality of arcuate segments corresponding to the relative volume of the lung lobe and bronchial segments, respectively.

In embodiments, the method further comprises determining a treatment modality based on the lung image information and a physician preference input.

In embodiments, the method further comprises treating the lung with an interventional appliance corresponding to said treatment modality from the determining step.

Still other descriptions, objects and advantages of the present invention will become apparent from the detailed description to follow, together with the accompanying drawings.

### BRIEF DESCRIPTION OF THE DRAWINGS

FIG. 1 is block diagram illustrating the components of a system in accordance with one embodiment of the invention.

FIG. 2 is a flow chart illustrating a method in accordance with one embodiment of the invention.

FIGS. 3A-3E are illustrations of graphical user interfaces for various categories of disease attributes.

FIG. 4 is an illustration of a graphical user interface for another main category corresponding to a physician technology preference.

FIG. 5 is an illustration graphically depicting patient data and output in accordance with one embodiment of the invention.

FIG. 6 is a flowchart illustrating a method in accordance with another embodiment of the invention; and

FIG. 7 is an illustration graphically depicting patient data and output in accordance with another embodiment of the invention

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail).

The following patents and applications are incorporated herein by reference in their entirety: U.S. Pat. Nos. 7,913,698; 8,585,645; 7,993,323; and U.S. Pat. Nos. 8,147,532; 8,734,380; and US Patent Publication No. 2015/0094607.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

System Overview

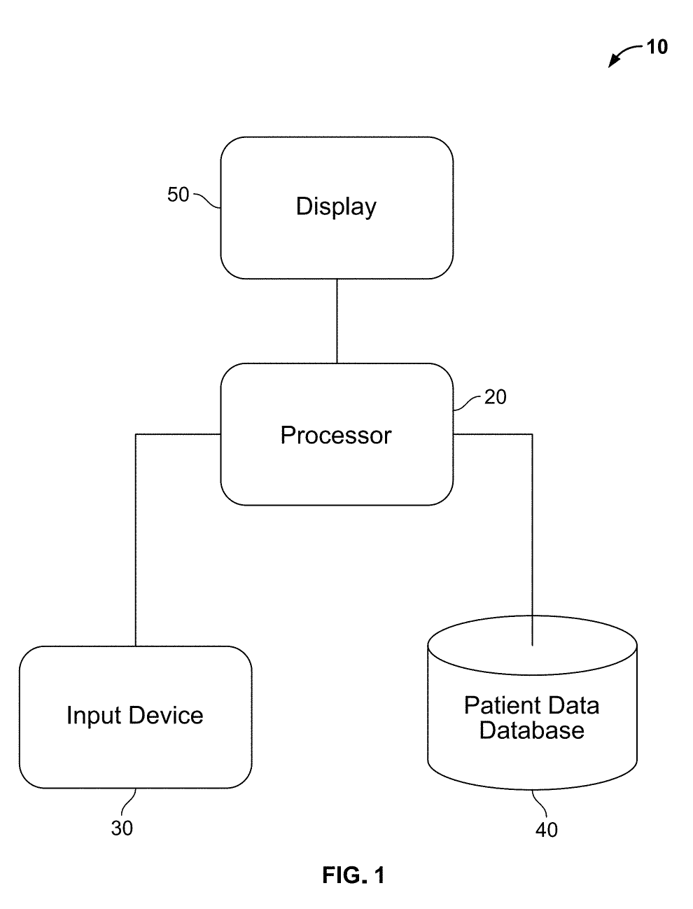

FIG. 1 is an overview of a system 10 in accordance with the present invention. System 10 is shown having a processor 20, which as described herein, is programmed and operable to assist a physician to determine an optimal treatment modality for lung volume reduction. Physician input is received by the processor via an input device 30 such as, for example, a mouse, keyboard, touchscreen display, etc.

System 10 is also shown comprising a database 40 of diagnostic information for one or more patients. The database may be stored locally, or on a remote server and linked to the processor through a wired, or wireless interface. Examples of diagnostic information include, without limitation, pulmonary function tests, diffusing capacity, perfusion, and results of CT quantitative analyses (fissure integrity, tissue volume, tissue density, disease severity).

System 10 is also shown including a display 50. Examples of displays include, without limitation, computer monitors, smart phone, tablet or PDA displays, etc. In embodiments, the input and output functionality are combined on a single touch screen display.

System 10 may be in the form of a desktop computer, laptop, tablet, smart phone, PDA or other apparatus and is only intended to be limited to a particular housing or configuration where recited in the appended claims.

Method Overview

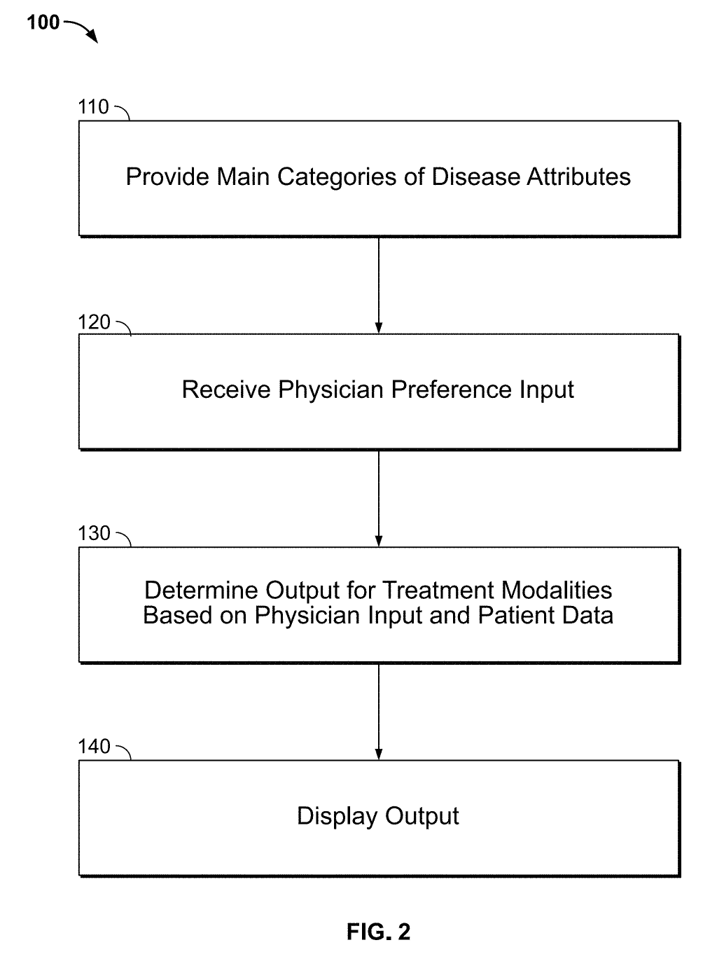

FIG. 2 is a flowchart illustrating an overview of a method 100 for assisting a physician determine an optimal treatment modality for lung volume reduction.

Step 110 states to provide a plurality of main categories of disease attributes. As described herein, various disease attributes may be displayed in a drop-down menu to the physician. Examples of disease attributes include, without limitation, heterogeneity, treatment location, treatment approach, maximum tissue destruction, and fissure integrity.

Step 120 states to receive the physician input. As described herein, a physician may input a preference input for each disease attribute described above.

Step 130 states to determine output corresponding to treatment modalities. In embodiments, and as described further herein, step 130 determines an optimal treatment modality or ranking of treatment modalities for one or more patient(s).

In embodiments, step 130 determines optimal treatment modalities for a percentage of patients based on the physician preference input for the disease attributes (main categories, and in some embodiments, sub-categories), and the patient data from the group of patients. As described herein the patient data may be entered by the physician, or received from an inter-connected electronic database of group data of patients. In embodiments, the physician enters the patient data.

Step 140 states to display output of the treatment modality. Output, as described herein, may displayed on a monitor or tablet device. Examples of output may include a preferred treatment modality, ranking, scores, or percentages of treatment modalities. In one embodiment, the output includes a ranking of modalities selected from the group consisting of vapor ablation, valves, coils, and DNQ (does not qualify or no LVR-type treatment).

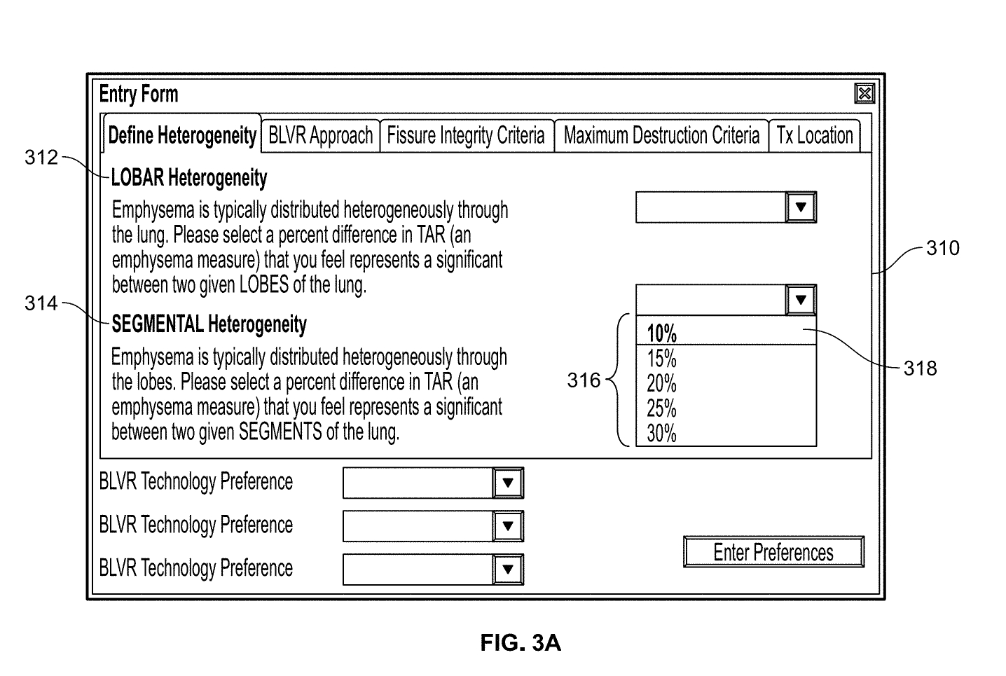

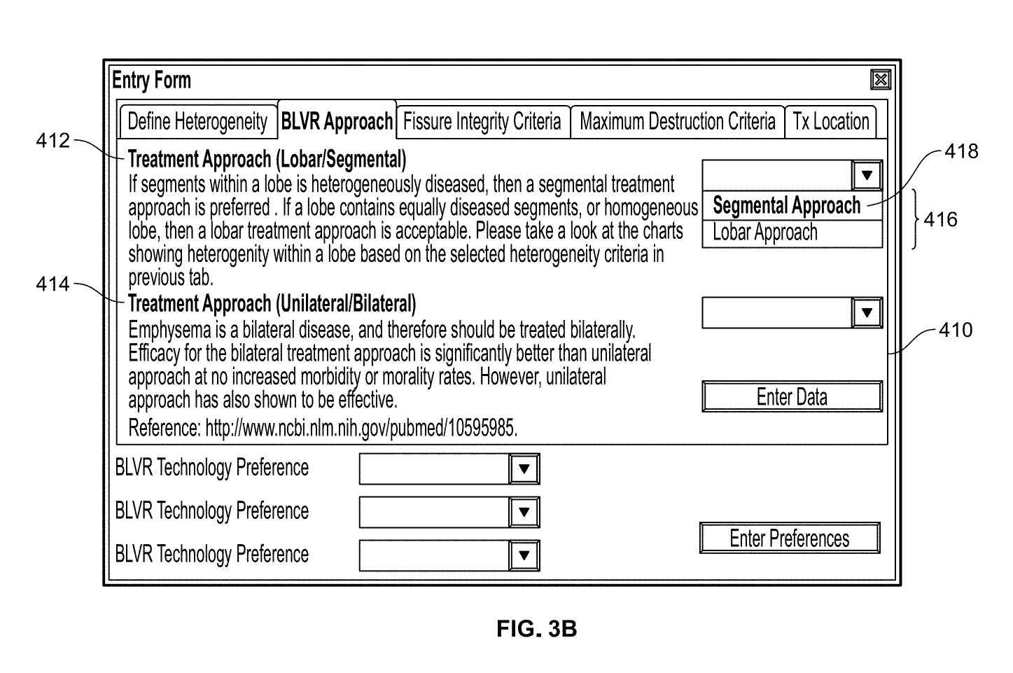

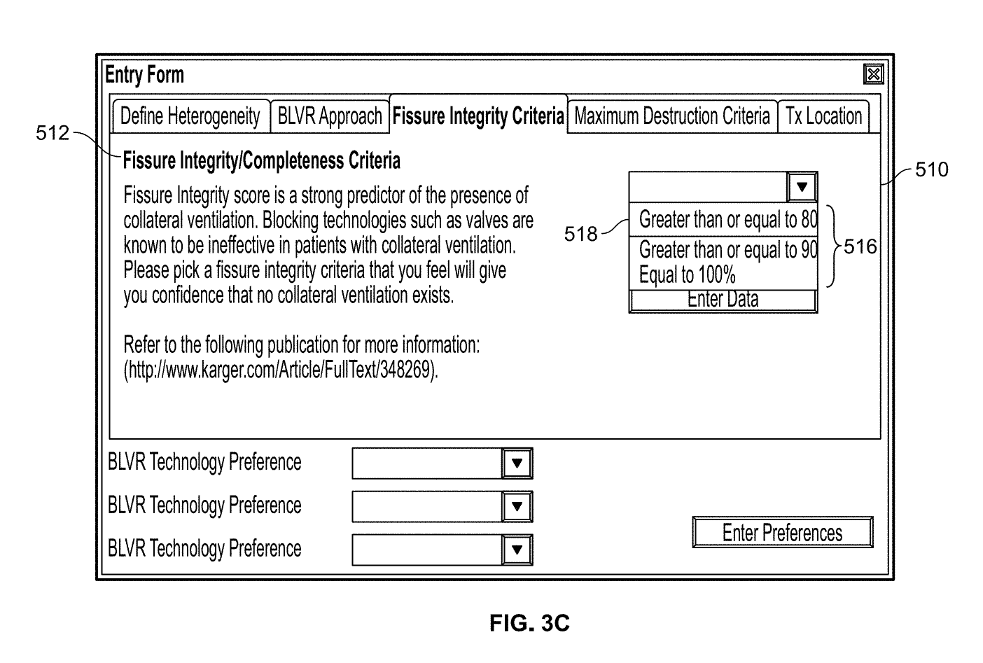

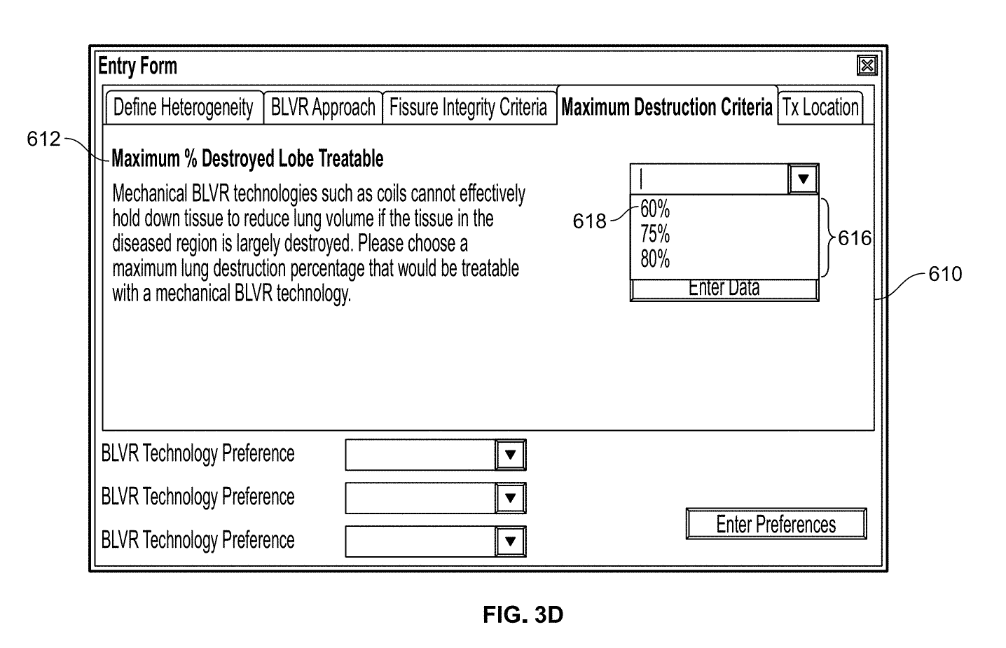

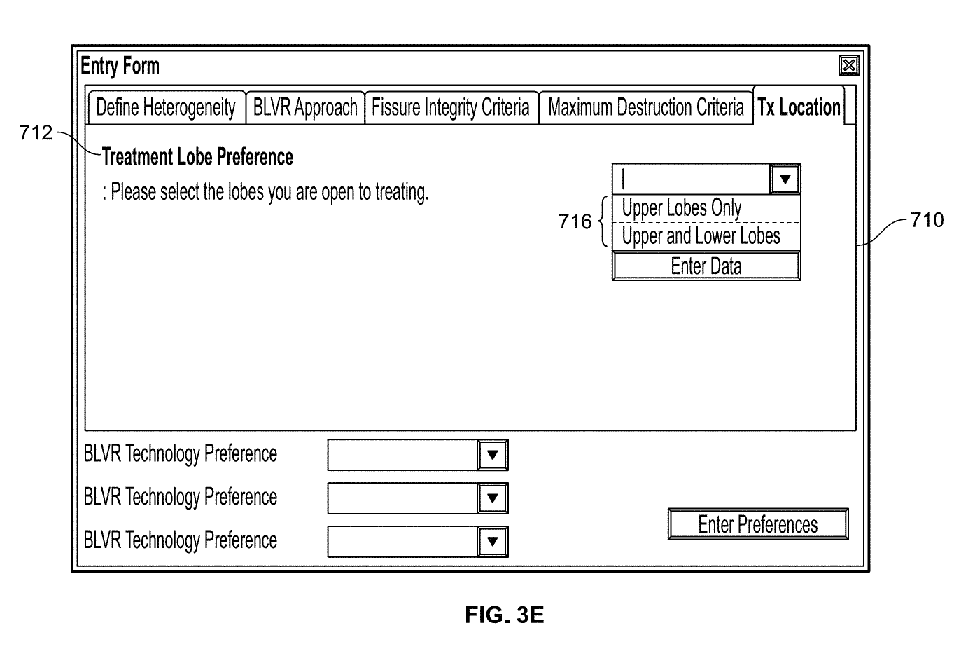

FIGS. 3A-3E are graphical displays of primary categories of disease attributes shown in tabular format. Examples of disease attributes include, without limitation, heterogeneity, treatment approach, fissure integrity, maximum tissue destruction, and treatment location.

Heterogeneity

FIG. 3A shows a graphical user interface to define heterogeneity 310 and to accept the physician's preference input 318. The embodiment illustrated in FIG. 3A includes two sub categories for heterogeneity including Lobar Heterogeneity 312 and Segmental Heterogeneity 314. In embodiments, a drop-down list of candidate physician input 316 is provided from which the physician may enter her likeness or preference input 318.

With reference to lobar heterogeneity 312, the physician enters the percent difference in the tissue density (e.g., without limitation, tissue to air ratio, mean lung density, and LAA %) that, in her view, represents a significant difference between two given lobes of the lung.

With reference to segmental heterogeneity 314, a drop-down menu or list of candidate inputs 318 is shown. The physician can select a significant density ratio 316 representing the density difference in two given segments of lung. In embodiments, the candidate inputs for segmental heterogeneity are density differences in the range of 10 to 30%.

In embodiments, instead of candidate inputs, the physician is prompted to enter any threshold value or amount. For example, a question or blank space is shown for the physician to respond to by entering the threshold value, percent, or amount.

Without intending to be bound by theory, one type of treatment may be excluded or preferred if there is a significant difference between the heterogeneity of the lobes or segments as described herein. For example, for highly heterogenous segments, a vapor ablation treatment may be preferred because a vapor ablation treatment can be segment specific. In contrast, valves are not segment specific because valves reduce an entire anatomy portion.

After the physician provides her preference input and the patient data is received, an optimal treatment modality is calculated using the physician inputs as thresholds to rank or exclude certain treatment modalities.

Treatment Approach

FIG. 3B shows a graphical user interface to define bronchoscopic lung volume reduction (BLVR) approach 410 and to accept the physician's preference input 418. The embodiment illustrated in FIG. 3B includes two sub categories including Lobar/segmental 412 and Unilateral/bilateral 414. In embodiments, a drop-down list of candidate physician input 416 is provided from which the physician may enter her preference input 418.

Without intending to be bound by theory, if segments within a lobe are heterogeneously diseased, then a segmental treatment approach is preferred. However, if a lobe contains equally diseased segments, or a homogeneous lobe, then a lobar treatment approach is acceptable. The physician selects a lobar or segmental approach. Additionally, in embodiments, the physician selects whether a unilateral or bilateral approach is preferred.

Fissure Integrity

FIG. 3C shows a graphical user interface to define fissure integrity 510 and to accept the physician's preference input 518. In embodiments, a drop-down list of candidate physician input 516 is provided from which the physician may enter her preference input 518.

Without intending to be bound by theory, fissure integrity score is a strong predictor of the presence of collateral ventilation. Lower fissure integrity is predictive of higher collateral ventilation. Blocking technologies such as valves are known to be ineffective in patients with high collateral ventilation. The physician chooses a fissure integrity criteria corresponding to a threshold value for collateral ventilation. The system allows the physician to qualify or disqualify certain technologies such as valve-based programs based on the level of collateral ventilation.

In embodiments, the preference input 518 is fissure integrity having a threshold value of at least 80%, 90%, and in some instances about 100%. A patient or group of patients having fissure integrity below this threshold value would be excluded from certain treatment modalities such as valves.

Maximum Destruction

FIG. 3D shows a graphical user interface to define maximum destruction criteria 610 and to accept the physician's preference input 618. In embodiments, a drop-down list of candidate physician input 616 is provided from which the physician may enter her preference input 618.

In embodiments, the physician selects the maximum percentage of destroyed lobes that they consider treatable.

Without intending to be bound by theory, mechanical BLVR technologies such as coils cannot effectively hold down tissue to reduce lung volume if the tissue in the diseased region is largely destroyed. In embodiments, the preference input is a maximum destruction criteria having a threshold value in the range from 60 to 80%. A patient or group of patients having tissue destruction above this threshold value would be excluded from certain treatment modalities such as coils.

Treatment Location

FIG. 3E shows a graphical user interface to define treatment location 710 and to accept a physician's preference input. In embodiments, a drop-down list of candidate physician input 716 is provided from which the physician may enter her preference input. In embodiments, the physician selects the anatomy or tissue locations that they consider treatable or reachable.

Without intending to be bound by theory, some locations may be too remote or difficult to reach for one physician or may not be approved for commercial use in a particular country, jurisdiction, or territory. In embodiments, a preference input is treatment location of the upper lobes only, outside of which certain technologies are excluded such as, e.g., vapor, which at the time of this filing only has approval for use in upper lobes in certain territories.

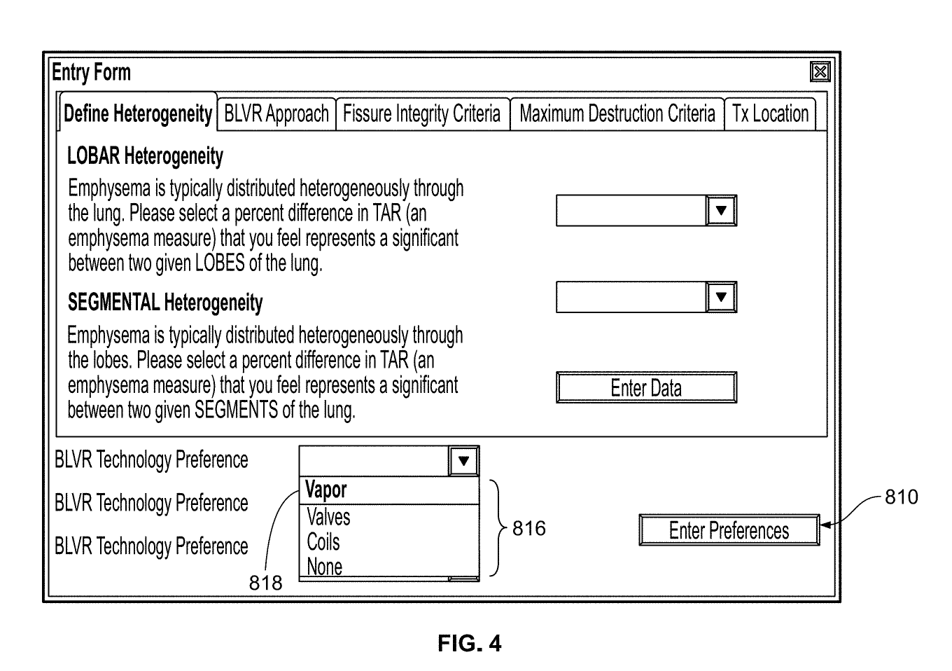

FIG. 4 shows a graphical user interface including BLVR physician technology preference 810. A set of candidate inputs 816 is provided as a drop-down menu, and the physician input 818 is selected. In the embodiment shown in FIG. 4 the physician input 818 corresponds to vapor or vapor ablation. Each physician may have a preference for one treatment modality over another, and in embodiments, the methods and systems described herein determine the optimal modality based, at least in part, on the physician technology preference 810. In embodiments, the physician preference for one type of technology 818 is used to overrule or prioritize one treatment modality over another if the treatment modalities are otherwise equal, substantially the same, or differ by a score of less than 10-15%.

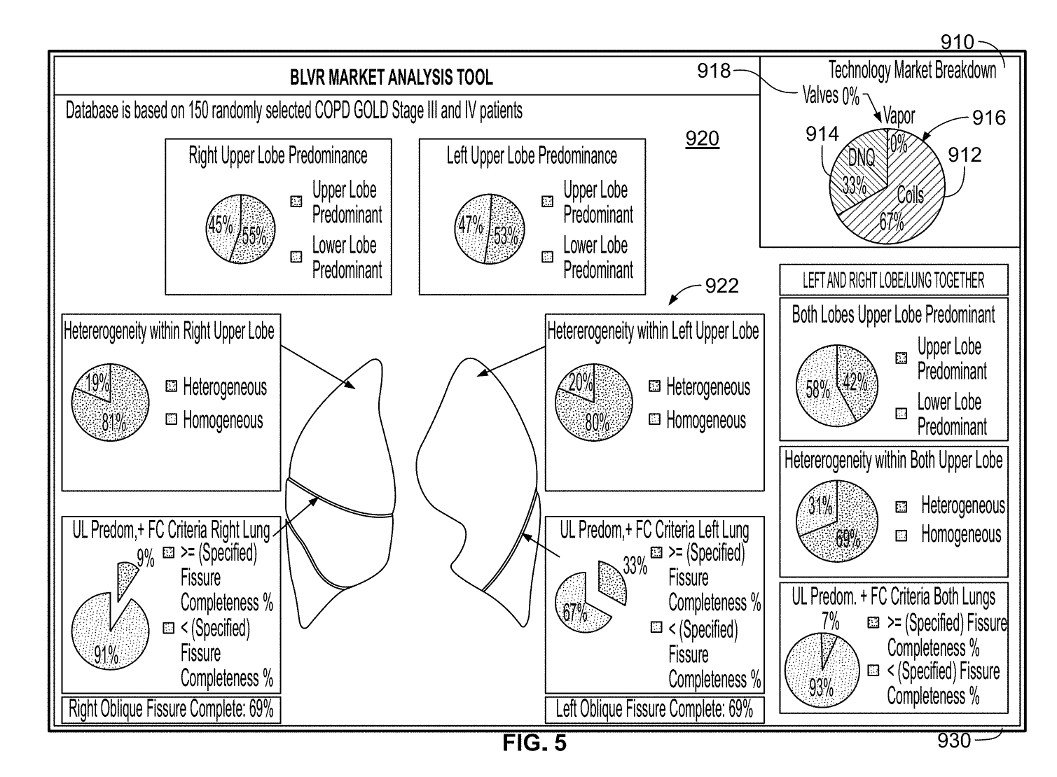

FIG. 5 illustrates output in accordance with embodiments of the invention. Graph 910 is an example output of a technology market breakdown showing vapor coils 67% (reference numeral 912), does not qualify (DNQ) 23% (reference numeral 914), and vapor and valves 0%. (916, 918 respectively). An indication for DNQ may be suggestive of do not treat with a BLVR procedure, much less a more invasive surgery such as surgical lung volume reduction. DNQ may also be suggestive of conservative care options such physical therapy or systemic treatment, or life style changes.

In embodiments, and although not shown, the output may include solely one modality as an optimal modality for a patient or group of patients based on the patient data and the inputs described herein. The output may be computed and shown as a listing, text line, text box, bar, or pie chart showing a score, percentage, icon, symbol, or color to indicate preference. Indeed, a wide range of text, data, results, and graphics may be displayed.

Physician inputs and patient data may also be graphically displayed as illustrated by the several charts 920. For example, the heterogeneity of the upper left lobe is shown in pie chart form 922 for the group of patients. In the embodiment illustrated in FIG. 5, the patient data includes one hundred fifty randomly selected COPD GOLD Stage III and IV patients.

Data relating to both lungs operating together as a whole is shown in pie charts 930.

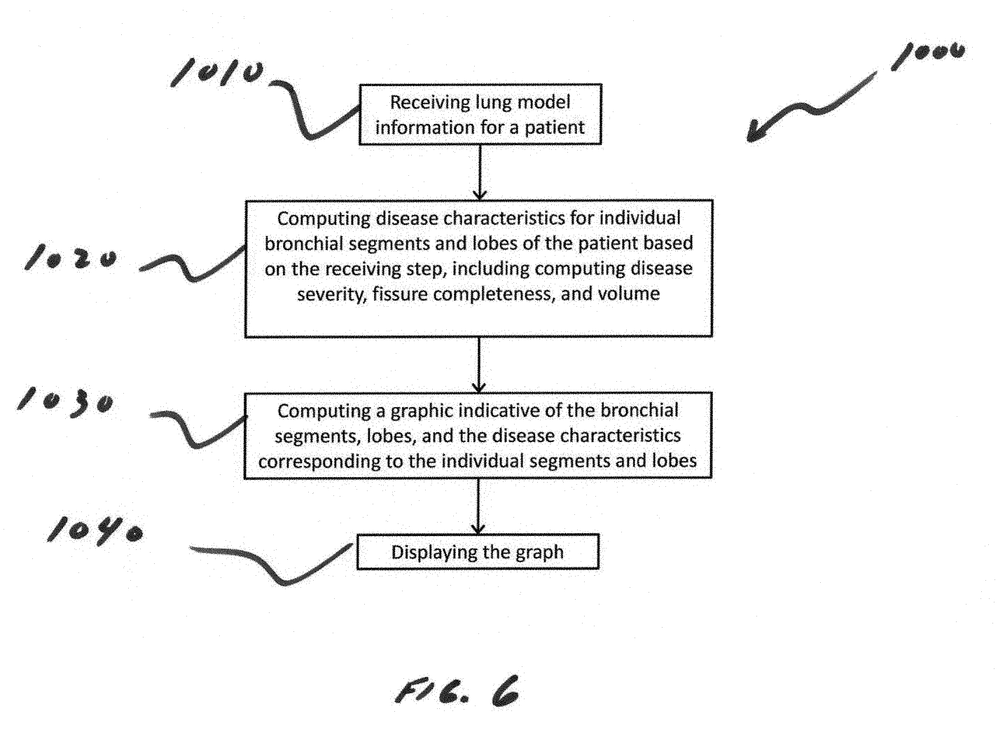

The patient data may be graphically displayed in a wide variety of ways. With reference to FIG. 6, for example, a method 1000 is shown for computing and graphically displaying patient data in accordance with another embodiment of the invention.

Step 1010 states to receive lung model information for a patient. Examples of lung model information include without limitation, various diagnostic information such as pulmonary function tests, diffusing capacity, perfusion, and image data such as CT image data.

Step 1020 states to compute disease characteristics for individual bronchial segments and lobes of the patient based on the receiving step, including fissure integrity, tissue volume, tissue density, disease severity. In embodiments, the disease severity is computed by correlating the voxel density with the disease, and higher density representing the higher presence of the emphysema. Additional examples of methods for determining disease severity are described in US Patent Publication No. 20120249546, and U.S. Pat. Nos. 9,390,498 and 9,504,529.

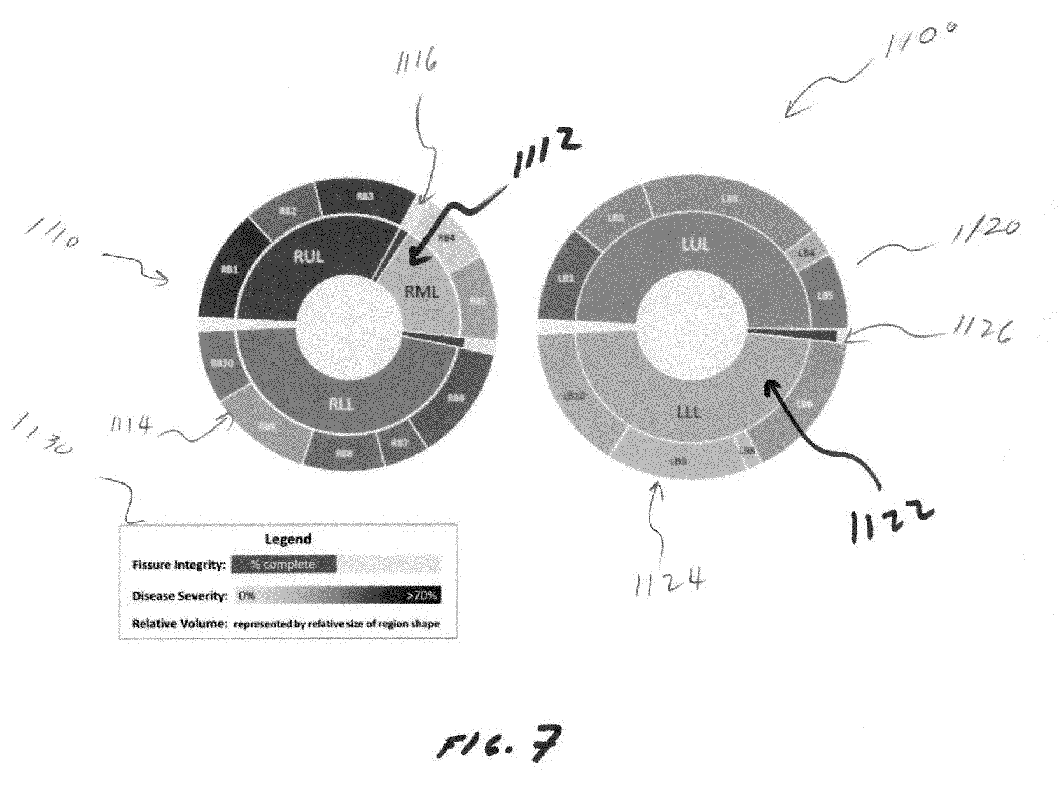

Step 1030 states to compute a graphic indicative of the bronchial segments, lobes, and the disease characteristics corresponding to the individual segments and lobes. An example of a graphic 1100 is shown in FIG. 7, discussed below.

Step 1040 states to display the graph. As described above, the step of displaying may be performed using a monitor, tablet, PDA, smart phone, screen, etc.

FIG. 7 illustrates a graphic 1100 in the form of a pair of donut charts 1110, 1120, and legend 1130.

Chart 1110 is representative of the right lung of a patient. Chart 1120 is representative of the left lung. Inner rings 1112, 1122 represent the lobes and outer rings 1114, 1124 represent the bronchial segments. The size of each segment or lobe shown in the rings corresponds to the relative volume of each segment or lobe in the lung of the patient.

With reference to legend 1130, relative shading of each segment or lobe corresponds to the disease severity. The darker the shading, the more severe the disease. Although shading is shown, the invention is not so limited. A wide range of colors, patterns, and/or other indicia may be provided to show disease severity.

Fissure integrity is also indicated by the presence of a break in the rings 1116, 1126, and the degree or completeness of the fissures or separation is indicated by the pattern or color shown in this break in the rings. The more complete the fissure, e.g., the break is shown as more full or nearly completely shaded. In contrast, the less complete the fissure, the break 1112, is shown partially full. Although shading is shown to fill the break, the invention is not so limited. A wide range of colors, patterns, and/or other indicia may be provided to show fissure completeness.

An advantage of one or more of the embodiments described herein is the improvement to the field of bronchoscopy, and more specifically, to BLVR. Bronchoscopy and BLVR are improved in meaningful ways because multiple diagnostic and physician preferences enable a physician to choose, restrict, rank and score various BLVR modalities for various patient population in an automatic, rigorous, consistent, and fast in real time manner based on patient quantifiable phenotype. This is a substantial improvement over previous techniques to eye-ball, gut-feeling, or speculation to which modality may be optimal. In accordance with embodiments discussed herein diagnostic data from multiple patients is applied and cross-examined with an individual physician for increased accuracy. It is anticipated that patient outcomes shall be improved with the present invention.

Although several embodiments have been disclosed above, it is to be understood that other modifications and variations can be made to the disclosed embodiments without departing from the subject invention. The invention includes any method, system, or apparatus for assisting a physician determine an optimal treatment modality for LVR, described herein, or any combination of features and steps described herein for determining an optimal treatment modality for LVR, described herein.

1. A system for assisting a physician perform a lung volume reduction procedure on a patient comprising:
a processor operable to:
(i) compute disease characteristics for bronchial segments, lung lobes, and lung fissures of the patient based on lung image information of the patient; and
(ii) transform the lung image information into a non-anatomical shaped graphic indicative of the relative volume and disease characteristics of the bronchial segments, lung lobes, and fissures of the patient; and
a display in communication with the processor and operable to present the graphic.

2. The system of claim 1, wherein graphic comprises a donut-shaped graphic for each lung.

3. The system of claim 2, wherein each donut-shaped graphic comprises an inner ring and an outer ring corresponding to the lung lobes and bronchial segments, respectively.

4. The system of claim 3, wherein each outer ring comprises a plurality of arcuate segments corresponding to the relative volume of each of the bronchial segments of the patient.

5. The system of claim 4, wherein each inner ring comprises a plurality of arcuate segments corresponding to the relative volume of each of the lung lobes of the patient.

6. The system of claim 5, wherein the processor is operable to compute a plurality radial segments extending through the inner and outer ring, and corresponding to the presence of a fissure in the patent lung.

7. The system of claim 6, wherein the processor is operable to compute disease severity and to show the level disease severity within each bronchial segment.

8. The system of claim 7, wherein the disease severity is shown by color, line pattern or fill, or shading.

9. The system of claim 6, wherein the processor is operable to transform the relative volume of the bronchial segments and lung lobes into arcuate segments having a size in proportion to the relative volumes of the bronchial segments and lung lobes.

10. The system of claim 9 wherein the processor is operable to compute disease severity and fissure integrity subsequent to the computation of relative volume, and to incorporate the disease severity and fissure integrity onto the donut-shaped graphics.

11. The system of claim 1, further comprising a housing within which the display and processor are located.

12. The system of claim 11, wherein the display is touchscreen type display.

13. The system of claim 1, further comprising an input device for receiving physician input preferences, and the processor further operable to compute a treatment modality based on the physician input, and the lung image information of the patient.

14. The system of claim 13, further comprising an interventional appliance corresponding to the treatment modality, for treating target areas of the patient lungs, and wherein the interventional appliance is selected from the group consisting a vapor ablation catheter, coil, and valve.

15. A method for assisting a physician perform a lung volume reduction procedure on a patient comprising:
computing disease characteristics for bronchial segments, lung lobes, and lung fissures of the patient based on received lung image information of the patient;
computing a non-anatomical shaped graphic indicative of the relative volume of the bronchial segments and lung lobes, and disease characteristics corresponding to the bronchial segments and the lung lobes of the patient; and
displaying the graphic.

16. The method of claim 15, wherein the step of computing the graphic is performed by transforming the image information of the patient's bronchial segments and lung lobes into the non-anatomical shaped graphic.

17. The method of claim 16, wherein the transforming comprises converting the lung image data for each lung into a donut-shaped graphic for each lung.

18. The method of claim 17, wherein each donut-shaped graphic comprises an inner ring and an outer ring, and wherein each inner and outer ring comprises a plurality of arcuate segments corresponding to the relative volume of the lung lobe and bronchial segments, respectively.

19. The method of claim 18, further comprising determining a treatment modality based on the lung image information and a physician preference input.

20. The method of claim 19, further comprising treating the lung with an interventional appliance corresponding to said treatment modality from the determining step.
